(19) United States
(12) Patent Application Publication
(10) Pub. No.: US 2013/0177483 A1
(43) Pub. Date: Jul. 11, 2013

(54) SCENT DISPENSER DEVICE

(71) Applicants: Robert William Lackey, Hickory, NC (US); Robert Charles Beckman, Vale, NC (US); Charles Beach Templeton, Matthews, NC (US); Jeri Lynn Lemke, Hickory, NC (US)

(72) Inventors: Robert William Lackey, Hickory, NC (US); Robert Charles Beckman, Vale, NC (US); Charles Beach Templeton, Matthews, NC (US); Jeri Lynn Lemke, Hickory, NC (US)

(73) Assignee: WEB PRODUCTS, LLC, Kansas City, MO (US)

(21) Appl. No.: 13/544,278

(22) Filed: Jul. 9, 2012

Related U.S. Application Data
(60) Provisional application No. 61/505,578, filed on Jul. 8, 2011.

(51) Int. Cl. A61L 9/14

(52) U.S. Cl. CPC A61L 9/14
USPC 422/123

(57) ABSTRACT

The present invention provides methods and systems for a scent dispenser device that includes a bottom portion having first and second pairs of spaced-apart, opposed sidewalls projecting that collectively form a cavity to define an upper edge of the device, a top portion that is selectively secured to the bottom portion, and at least one opening in the top portion that allows air to flow through the device.

### CROSS REFERENCE TO RELATED PATENT APPLICATION

The current application claims the benefit of the earlier priority filing date of the provisional application Ser. No. 61/505,578, that was filed on Jul. 8, 2011.

### FIELD OF THE INVENTION

The present invention relates generally to a scent dispenser device and more generally relates to a scent dispenser device that contains a scent panel disposed within a bottom portion, wherein the bottom portion may be engaged to a filter, HVAC ductwork, register grill, or vent.

### BACKGROUND OF THE INVENTION

Many homeowners routinely utilize a spray to neutralize odors or an apparatus to provide a scented fragrance into the habitable space of their homes. The sprays to neutralize odors are limited to a short amount of time and for a limited space. The spray is not continuous and limited in area. For a scented fragrance, candles were typically used to transmit the scent into the air, but today, a number of devices are marketed heavily to provide scented air. However, these devices only provide the scent to the immediate area and for a limited amount of time, such as an hour with a candle. There is a need to provide a device to provide scent to a home that has a prolonged life and can be used throughout the residence. It is an object of the present invention to create a device that may be attached to either an air filter or HVAC ductwork for reducing odor and infusing the air passing through the filter or ductwork with a scent.

### BRIEF SUMMARY OF THE INVENTION

According to an embodiment of the present invention, the present invention is a scent dispenser device that includes a bottom portion having first and second pairs of spaced-apart, opposed sidewalls that form a cavity to define an upper edge of the device, a top portion that is selectively secured to the bottom portion, and at least one opening in the top portion that allows air to flow through the device.

According to another embodiment of the present invention, a scent dispenser device that includes a bottom portion that has an opening for allowing air to flow through the cavity of the bottom portion and through the at least on opening in the top portion.

According to yet another embodiment of the present invention, a scent dispenser device that includes a plurality of openings in the top portion for allowing air to flow through the device.

According to yet another embodiment of the present invention, a scent dispenser device that includes a rim extending outwardly from the sidewalls.

According to yet another embodiment of the present invention, a scent dispenser device that includes at least one tab that is resiliently flexible for allowing the device to be engaged to an air filter.

According to yet another embodiment of the present invention, a scent dispenser device that includes a top portion that is a scent panel with scent additives molded into the top portion.

According to yet another embodiment of the present invention, a scent dispenser device that includes a scent panel composed of polyethylene.

According to yet another embodiment of the present invention, a scent dispenser device that includes a front slot disposed on the device for receiving a latch disposed on the top portion, wherein the top portion is selectively engaged to the bottom portion.

According to yet another embodiment of the present invention, a scent dispenser device that includes a bottom portion having first and second pairs of spaced-apart, opposed sidewalls that collectively form a cavity to define an upper edge of the device. A top portion that is selectively secured to the bottom portion and a latch is disposed on the top portion for selectively engaging the top portion to the bottom portion. A plurality of openings in the top portion that allow air to flow through the device.

According to yet another embodiment of the present invention, a scent dispenser device that includes a top portion that has a front side, a back side, a left side, and a right side and a pair of flanges extending outwardly from the back side for selectively securing the top portion to the bottom portion and a latch is disposed on the front side of the top portion.

According to yet another embodiment of the present invention, a scent dispenser device that includes ridges disposed on the bottom portion of the bottom portion of the device for gripping an air filter.

According to yet another embodiment of the present invention, a scent dispenser device that includes at least one resiliently flexible tab containing a bore for receiving an attachment means for attaching the device to a structure and in the stream of air flow.

According to yet another embodiment of the present invention, a scent dispenser device that includes a pair of rear slots within the bottom portion for receiving a pair of flanges disposed on the top portion, wherein the pair of flanges are inserted into the pair of rear slots such that the pair of flanges are easily removed from the pair of rear slots and allow the top portion to be selective secured to the bottom portion.

According to yet another embodiment of the present invention, a scent dispenser device that includes a bottom portion having first and second pairs of spaced-apart, opposed sidewalls that collectively form a cavity to define an upper edge of the device, and at least one rear slot disposed on a sidewall and a front slot disposed on a sidewall. A top portion having at least one rear flange extending outwardly from the top portion for insertion into the at least one rear slot and a latch disposed on the top portion for insertion into the front slot, wherein the top portion is selectively secured to the bottom portion. A plurality of openings in the top portion that allows air to flow through the device.

According to yet another embodiment of the present invention, a scent dispenser device that includes a shelf positioned in close proximity to the front slot and at least one protrusion disposed on the latch, wherein the at least one finger is engaged to the shelf.

### BRIEF DESCRIPTION OF THE DRAWINGS

The present invention is illustrated and described herein with reference to the various drawings, in which like reference numbers denote like method steps and/or system components, respectively, and in which:

FIG. 1 is a perspective view of the scent dispenser device;

FIG. 2 is a perspective view of the scent dispenser device and scent media;

FIG. 3 is a perspective view of the scent dispenser device engaged to an air filter;

FIG. 4 is a perspective rear view of the scent dispenser device;

FIG. 5 is a perspective view of the bottom portion of the scent dispenser device; and

FIG. 6 is a perspective view of the latch of the top portion of the scent dispenser device.

### DETAILED DESCRIPTION OF THE INVENTION

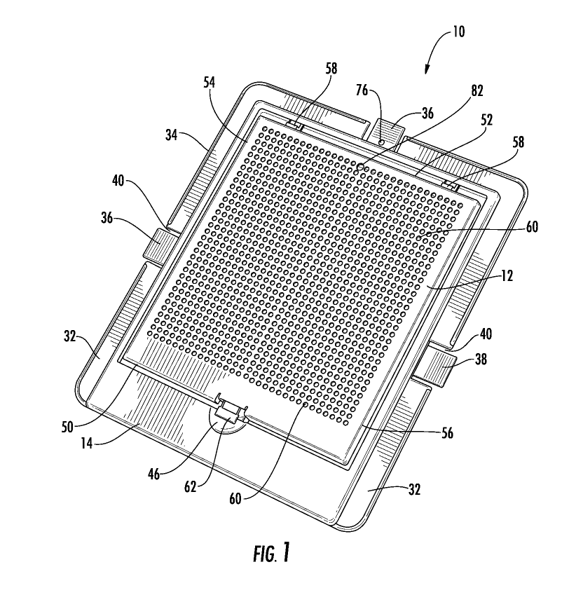

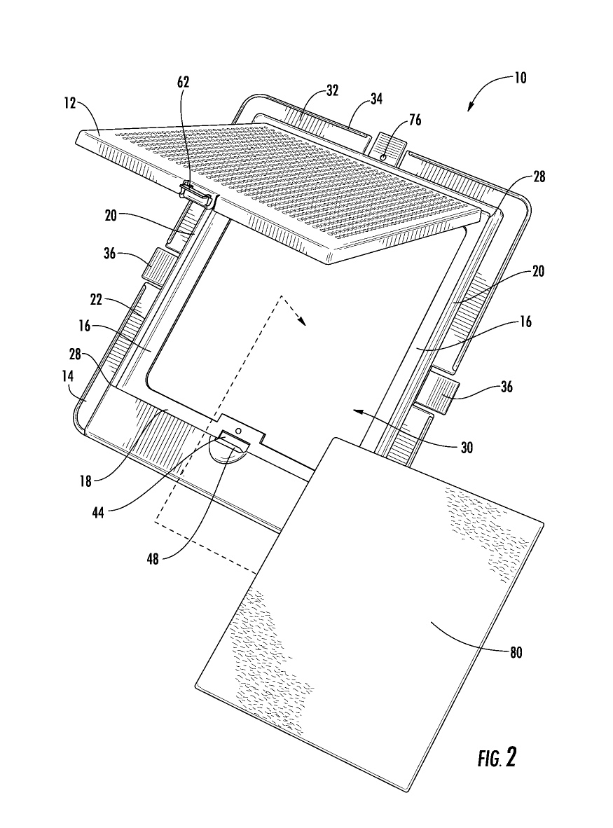

Referring now specifically to the drawings, a scent dispenser device is illustrated in FIGS. 1-5 and is shown generally at reference numeral 10. The device 10 includes a top portion 12 and a bottom portion 14. The bottom portion 14 includes an outer edge 16. First and second pairs of opposed sidewalls 18, 20 extend from the outer edge 16 to an upper edge 22. The sidewalls 18, 20 each have inner and outer sidewall surfaces 24, 26. As shown in FIG. 1, each of the second pair of sidewalls 20 interconnects the first pair of sidewalls 18 to define corners 28. The intersection of the second pair of sidewalls 20 and first pair of sidewalls 18 also defines a cavity 30 therein. A rim 32 extends outwardly from the outer sidewall surfaces 26 and the upper edge 22.

The rim 32 extends outwardly from the outer sidewall surfaces 26. As illustrated in FIGS. 1-5, the rim 32 extends outwardly from the outer sidewall surfaces on a plane that is offset from the upper edge 22 on three of the four sidewalls 18, 20. On the fourth sidewall 20, the rim 32 is substantially planar with the upper edge 22. In other words, the rim 32 extends outwardly and is in a planar relationship with the outer edge 16 on three of the four sidewalls 18, 20 and the fourth sidewall 20 contains a rim 32 that is in a planar relationship with the upper edge 22. The rim 32 includes a first end and a second end, wherein the first end of the rim 32 is disposed near the sidewall 18, 20 and the second end is opposite the first end. A lip 34 extends substantially perpendicularly upward from the second end of the rim 32.

As illustrated in FIGS. 1-5, at least one tab 36 is disposed within the rim 32. The tab 36 has an upper portion, a lower portion, a front side, and a back side. The tab 36 has an upper portion disposed in close proximity to the first end of the rim 32. At least one notch 38 is disposed on the front side of the lower portion. The tab 36 is resiliently flexible and flexes when a small amount of pressure is exerted on the tab 36. Preferably, the tab 36 includes a plurality of ridges 38 disposed on the front side of the lower portion. The sides of the tab 36 are spaced-apart from the rim 32, thus allowing the tab 36 to flex or move in the forward and backward direction while still being engaged to the rim 32. Preferably, a slit 40 is positioned between the tab 36 and rim 32, allowing the tab 36 to flex or move unobstructed by the rim 32. The tabs 36 are contained on three of the four sidewalls 18, 20.

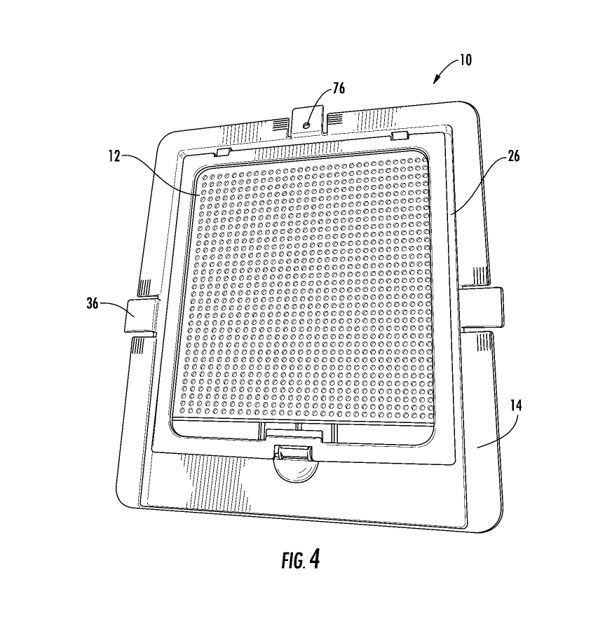

As illustrated in FIG. 4, at least one ridge 38 is disposed on the back side of the rim 32. The ridge 38 is disposed near the lower portion of the back side of the rim 32. As illustrated, a plurality of ridges 38 are disposed on the back side of the rim 32 and on either side of the tab 36. The ridges 38 are positioned on either side of the tab 36, and an identical number of ridges 38 are positioned on either side of the tab 36 and spaced apart from one another.

At least one rear slot 42 is positioned on one of the opposed sidewalls 18, 20 and extends from the inner sidewall surface 24 to the outer sidewall surface 26. The bottom portion of the rear slot 42 is preferably adjacent the outer edge 16. The upper portion of the rear slot 42 is positioned along the length of the sidewalls 18, 22 on which it is positioned. The device 10 preferably contains two spaced-apart rear slots 42, containing the above described features. The two spaced-apart rear slots 42 are positioned on one of the opposed sidewalls 18, 22 and extend from the inner sidewall surface 24 to the outer sidewall surface 26. The bottom portion of the rear slot 42 is preferably adjacent the outer edge 16. The upper portion of the rear slot 42 is positioned along the length of the sidewalls 18, 22 on which it is positioned. The two rear slots 42 are spaced on either side of the tab 36 that is positioned on the rim 32 external to the two rear slots 42.

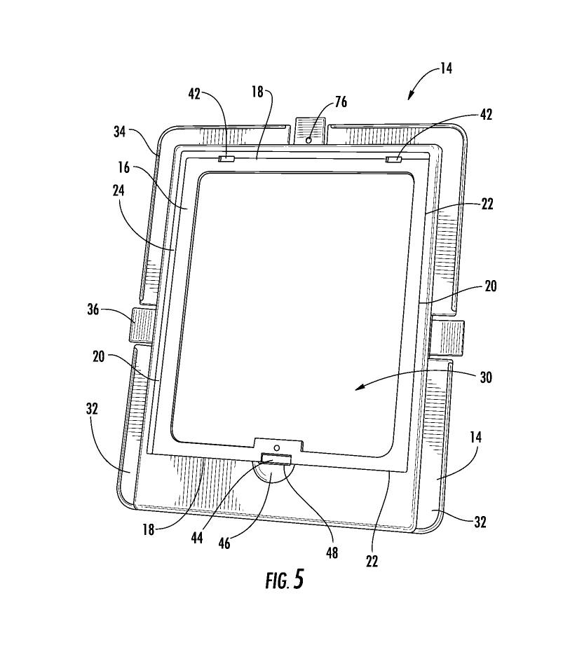

The outer edge 16 contains a front slot 44 disposed on the rim 32. Preferably, this front slot 44 is positioned opposite the at least one rear slot 42. For example, the at least one rear slot 42 is positioned on sidewall 18, and the front slot 44 is positioned on the outer edge 16 adjacent opposed sidewall 18. Alternatively, the at least one front slot 44 is positioned on sidewall 20, and the front slot 44 is positioned on the opposed sidewall 20. The area where the front slot 48 is located contains a depression 46 formed within the upper edge 22 and rim 32. An edge of the depression 46 contains a shelf 48. As illustrated in FIG. 5, the depression has a semi-circle shape that extends into the rim 32. In other words, the front slot 44 is positioned lower than the upper edge 22 surrounding the device 10. As illustrated in FIG. 5, the shelf 48 spans substantially the length of the front slot 44. However, the shelf 48 may be of a length and width that is suitable for its intended purposes.

The top portion 12 of the device 10 is designed to fit within the cavity 30 and is selectively secured to the bottom portion 14. The top portion 12 contains a front side 50, back side 52, left side 54, and right side 56. The back side 52 of the top portion 12 contains two flanges 58 that extend outwardly from the top portion 12

The top portion 12 contains at least one opening 60, and as illustrated in FIGS. 1-4, the top portion contains a plurality of openings 60 that are spaced-apart and equal distance from each other. Additionally, the openings 60 are of the same diameter and are arranged having a symmetrical pattern. In an alternative embodiment, the openings 60 may have any shape, size, or diameter and arranged in any pattern or arrangement so as to accomplish the plurality of openings 60 intended function. Alternatively, the top portion 12 of the device 10 may contain a single opening 60, have two openings 60, or contain a plurality of openings 60.

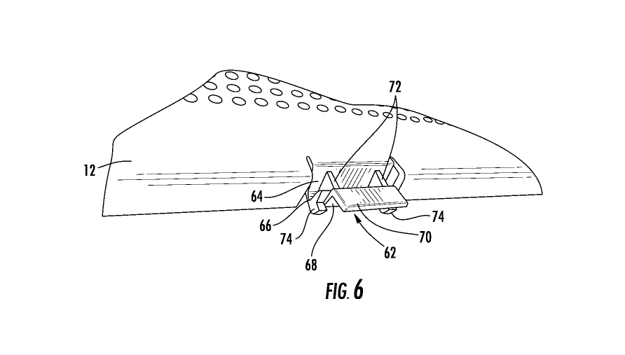

The front side 50 of the top portion 12 contains a latch 62 is disposed thereon. The latch 62 is designed to be received within the front slot 44. Any latch 62 may be utilized that would allow the top portion 12 to be selectively secured to the bottom portion 14. As illustrated in FIG. 6, the latch 62 consists of a first vertical portion 64 that is engaged to a horizontal portion 66 that is engaged to the second vertical portion 68. A finger 70 extends from the second vertical portion 68 and allows a user to disengage the latch 62 from the front slot 44. The horizontal portion 66 creates a spaced-apart relationship between the first vertical portion 64 and the second vertical portion 68. A pair of braces 72 may be engaged to the first vertical portion 64 and the second vertical portion 68. The second vertical portion 68 contains a protrusion 74 or protrusions 74 that are designed to engage the shelf 48 of the front slot 44. The protrusions 74 are engaged to the shelf 48 by friction fit.

When a user desires to raise the top portion 12 of the device 10, the user lifts the finger 70. As pressure is applied to the finger 70 by the user, the protrusions 74 disengage the shelf 48, allowing the user to pull the top portion 12 upward and separate the top portion 12 from the bottom portion 14.

The top portion 12 may be composed of a low density polyethylene or high density polyethylene base resin that is blended with ethylene-vinyl acetate. A scented oil, preferably with a concentration of up to 30% is included to provide the scent. The bottom portion 14 of the device 10 may be composed of polystyrene or polypropylene. However, one of ordinary skill in the art will understand the top portion 12 and bottom portion 14 may be composed of any material suitable for its uses.

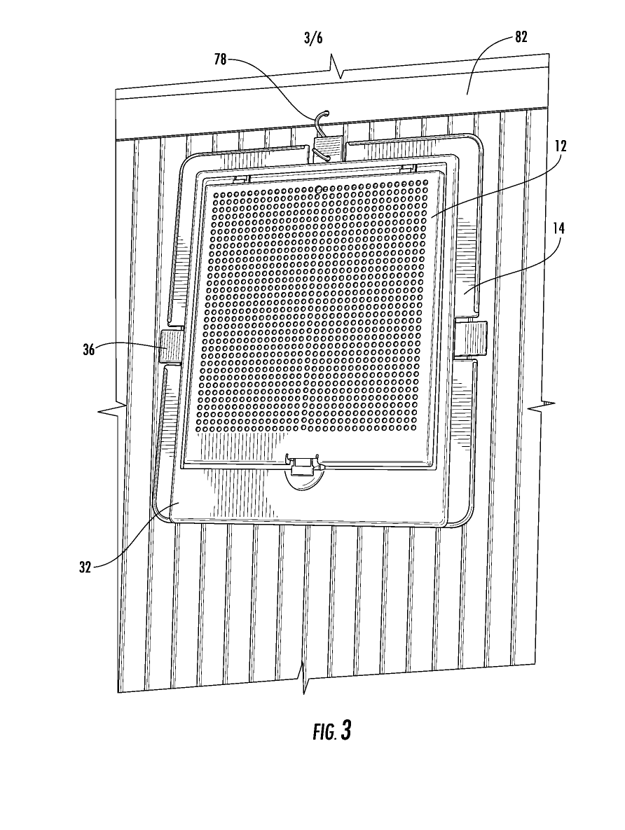

During use, the device 10 is placed within the stream of air so that the air can pass through the cavity 30 and openings 60. The device 10 may be engaged to an air filter, HVAC ductwork, register, grill, vent or the like. The tabs 36 are designed to be engaged to the frame of an air filter. The filter frame is placed within the slit 40, allowing the tab 36 to be placed behind the filter frame. The ridges 38 on the tab 36 engage the filter frame, and likewise, the ridges 38 of the rim 32 are engage to the filter frame. A tab 36 may also contain a bore 76 for receiving a hook 78. As illustrated in FIG. 3, the hook 78 is an s-shaped hook that is has a first end and a second end, whereby the first end of the hook 78 is received within the bore 76 and the second end is engaged to the filter, HVAC ductwork, register, grill, vent or the like for positioning the device 10 within the stream of air flow. Alternatively, the hook 78 may be engaged to a bore 82 located within the top portion 12 for engaging only the top portion 12 to the filter, HVAC ductwork, register, grill, vent or the like for positioning the top portion 12 within the stream of air flow.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention and are intended to be covered by the following claims.

What is claimed is:

1. A scent dispenser device, comprising:
a bottom portion with first and second pairs of spaced-apart, opposed sidewalls that collectively form a cavity to define an upper edge of the device;
a top portion that is selectively secured to the bottom portion; and
at least one opening in the top portion that allows air to flow through the device.

2. The scent dispenser device of claim 1, further comprising a bottom portion that has an opening for allowing air to flow through the bottom portion and through the at least one opening in the top portion.

3. The scent dispenser device of claim 1, further comprising a plurality of openings in the top portion for allowing air to flow through the device.

4. The scent dispenser device of claim 1, further comprising a rim extending outwardly from the sidewalls.

5. The scent dispenser device of claim 1, further comprising at least one tab that is resiliently flexible for allowing the device to be engaged to an air filter.

6. The scent dispenser device of claim 1, wherein the top portion is a scent panel with scent additives molded into the top portion.

7. The scent dispenser device of claim 1, wherein the top portion is composed of polyethylene.

8. The scent dispenser device of claim 1, further comprising a front slot disposed on the device for receiving a latch disposed on the top portion, wherein the top portion is selectively engaged to the bottom portion.

9. A scent dispenser device, comprising:
a bottom portion with first and second pairs of spaced-apart, opposed sidewalls that collectively form a cavity to define an upper edge of the device;
a top portion that is selectively secured to the bottom portion;
a latch disposed on the top portion for selectively engaging the top portion to the bottom portion; and
a plurality of openings in the top portion that allows air to flow through the device.

10. The scent dispenser device of claim 9, wherein the top portion has a front side, a back side, a left side, and a right side and a pair of flanges extending outwardly from the back side for selectively securing the top portion to the bottom portion and a latch disposed on the front side of the top portion.

11. The scent dispenser device of claim 9, further comprising ridges disposed on the bottom portion of the device for gripping an air filter.

12. The scent dispenser device of claim 9, further comprising at least one resiliently flexible tab for attaching the device to an air filter.

13. The scent dispenser device of claim 9, further comprising at least one resiliently flexible tab containing a bore for receiving an attachment means for attaching the device to a structure and in the stream of air flow.

14. The scent dispenser device of claim 9, further comprising a pair of rear slots within the bottom portion for receiving a pair of flanges disposed on the top portion, wherein the pair of flanges are inserted into the pair of rear slots such that the pair of flanges are easily removed from the pair of rear slots and allow the top portion to be selective secured to the bottom portion.

15. A scent dispenser device, comprising:
a bottom portion having an outer edge with first and second pairs of spaced-apart, opposed sidewalls projecting from the outer edge to collectively form a cavity to define an upper edge of the device, and at least one rear slot disposed on the bottom portion and a front slot disposed on the bottom portion;
a top portion having at least one rear flange extending outwardly from the top portion for insertion into the at least one rear slot and a latch disposed on the top portion for insertion into the front slot, wherein the top portion is selectively secured to the bottom portion; and
a plurality of openings in the top portion that allows air to flow through the device.

16. The scent dispenser device of claim 15, wherein the top portion is a scent panel with scent additives molded into the top portion.

17. The scent dispenser device of claim 15, further comprising a rim extending outwardly from the upper edge and containing at least one resiliently flexibly tab positioned on the rim for engagement to an air filter.

18. The scent dispenser device of claim 15, further comprising ridges disposed on the bottom portion of the device for gripping an air filter.

19. The scent dispenser device of claim 15, further comprising a shelf positioned in close proximity to the front slot and at least one protrusion disposed on the latch, wherein the at least one finger is engaged to the shelf.

20. The scent dispenser device of claim 15, wherein the top portion is composed of polyethylene with scent additives molded into the polyethylene.
